(19) United States
(12) Patent Application Publication
(10) Pub. No.: US 2023/0063703 A1
(43) Pub. Date: Mar. 2, 2023

(54) DRUG AND FOOD FOR TREATING AND/OR PREVENTING ENTERITIS AND/OR HEPATITIS

(71) Applicants: Keio University, Minato-ku, Tokyo (JP); Kagami Inc., Ibaraki-shi, Osaka (JP)

(72) Inventors: Takanori KANAI, Tokyo (JP); Tomohisa SUJINO, Tokyo (JP); Satoko UMEDA, Tokyo (JP); Kentaro MIYAMOTO, Tokyo (JP)

(73) Assignees: Keio University, Minato-ku, Tokyo (JP); Kagami Inc., Ibaraki-shi, Osaka (JP)

(21) Appl. No.: 17/800,306

(22) PCT Filed: Feb. 18, 2021
(86) PCT No.: PCT/JP2021/006227

§ 371 Date: Aug. 17, 2022

(30) Foreign Application Priority Data
Feb. 18, 2020 (JP) 2020-024984

(51) Int. Cl. A61K 31/197; A23L 33/175; A61P 29/00; A61P 1/04; A61P 1/16; A61P 31/14

(52) U.S. Cl. CPC A61K 31/197; A23L 33/175; A61P 29/00; A61P 1/04; A61P 1/16; A61P 31/14

(57) ABSTRACT

The present invention provides a drug for treating and/or preventing enteritis and/or hepatitis, said drug containing D-amino acid as an active ingredient. In addition, the present invention provides a food composition for suppressing enteritis and/or hepatitis, said composition containing D-amino acid as an active ingredient. In addition, the present invention provides a method for assisting diagnosis of enteritis and/or hepatitis in a subject, said method using the amount of D-amino acid in the intestinal tract as an indicator.

### FIELD

The invention relates to a drug and food for treatment and/or prevention of enteritis and/or hepatitis. The invention further relates to a method for assisting diagnosis of enteritis and/or hepatitis in a patient.

### BACKGROUND

It was once thought that only L-amino acids are involved in the constitution of organisms, but it has been shown that D-amino acids are also present in trace amounts, and that they have physiological functions and are associated with various diseases. For example, it has been reported that in the brain, D-Ser binds to N-methyl-D-Aspartate (NMDA) receptors in the central nervous system, exhibiting physiological effects (NPL 1).

Enterobacteria have also been reported to synthesize D-amino acids. For example, it has been reported that D-Ser synthesized by enterobacteria reach the kidneys through the blood, alleviating kidney disorders (NPL 2). It has also been reported that D-amino acid oxidizing enzyme (DAO) secreted from the host intestinal tract decomposes D-amino acids synthesized by enterobacteria, the hydrogen peroxide secreted during the procedure inhibiting proliferation of intestinal pathogenic bacteria (NPL 3).

PTL 1 also reports that extract from Lactobacillus bacteria has an anti-inflammatory effect, and that D-amino acids (such as D-alanine, D-serine, D-glutamic acid and D-aspartic acid) are present in Lactobacillus bacterial extract (PTL 1). None of these publications, however, report that the D-amino acids themselves can directly serve as drugs or foods for treatment and/or prevention of enteritis and/or hepatitis, nor do they even teach that D-amino acids can be indicated as active ingredients for drugs or foods.

### CITATION LIST

### Patent Literature

[PTL 1] International Patent Publication No. WO2010/027344

### Non Patent Literature

[NPL 1] FEBS Journal 275 (2008) 3514-3526.
[NPL 2] JCI Insight. 2018; 3 (20):e97957.
[NPL 3] Nature Microbiology vol. 1(2016), Article number: 16125.

### SUMMARY

### Technical Problem

It is an object of the present invention to provide a novel drug and food for treatment and/or prevention of enteritis and/or hepatitis. It is another object to provide a method for assisting diagnosis of enteritis and/or hepatitis.

### Solution to Problem

A 5-aminosalicylic acid formulation improves symptoms of inflammatory bowel disease by an active oxygen removal effect and leukotriene production inhibiting effect.

As a result of avid research, the present inventors have newly found that D-amino acids that produce hydrogen peroxide in the intestinal tract unlike 5-aminosalicylic acid formulations, exhibit an effect of inhibiting enteritis and/or hepatitis.

As a result of further research, the present inventors have also newly found that the amount of D-amino acids in the intestinal tract can serve as an index for diagnosis of enteritis and/or hepatitis.

The present invention includes the following [1] to [7H].

[1] A drug for treatment and/or prevention of enteritis and/or hepatitis, which comprises a D-amino acid, or its modified form or derivative, or a pharmaceutically acceptable salt thereof, as an active ingredient.

[2] A drug according to [1] above, wherein the D-amino acid is one or more selected from the group consisting of D-Ala, D-Pro, D-Gln, D-Val, D-Leu, D-Phe, D-Lys, D-Trp, D-Glu, D-Ser and D-Asp.

[3] A drug according to [1] or [2] above, wherein the enteritis is inflammatory bowel disease.

[4] A drug according to [3] above, wherein the inflammatory bowel disease is ulcerative colitis.

[5] A drug according to any one of [1] to [4] above, wherein the hepatitis is cholangitis.

[6] A drug according to [5] above, wherein the cholangitis is primary sclerosing cholangitis (PSC).

[1A] A food composition for inhibition of enteritis and/or hepatitis, which comprises a D-amino acid, or its modified form or derivative, or a food-acceptable salt thereof, as an active ingredient.

[2A] A food composition for inhibition of enteritis and/or hepatitis according to [2A] above, wherein the D-amino acid includes one or more selected from the group consisting of D-Ala, D-Pro, D-Gln, D-Val, D-Leu, D-Phe, D-Lys, D-Trp, D-Glu, D-Ser and D-Asp.

[3A] A food composition according to [1A] or [2A] above, wherein the enteritis is inflammatory bowel disease.

[4A] A food composition according to [3A] above, wherein the inflammatory bowel disease is ulcerative colitis.

[5A] A food composition according to any one of [1A] to [4A] above, wherein the hepatitis is cholangitis.

[6A] A food composition according to [5A] above, wherein the cholangitis is primary sclerosing cholangitis (PSC).

[7A] A food comprising a food composition according to any one of [1A] to [6A] above.

[1B] A D-amino acid, or its modified form or derivative, or a pharmaceutically acceptable salt thereof, to be used for treatment and/or prevention of enteritis and/or hepatitis.

[2B] A D-amino acid, or its modified form or derivative, or a pharmaceutically acceptable salt thereof according to [1B] above, wherein the D-amino acid includes one or more selected from the group consisting of D-Ala, D-Pro, D-Gln, D-Val, D-Leu, D-Phe, D-Lys, D-Trp, D-Glu, D-Ser and D-Asp.

[3B] A D-amino acid, or its modified form or derivative, or a pharmaceutically acceptable salt thereof according to [1B] or [2B] above, wherein the enteritis is inflammatory bowel disease.

[4B] A D-amino acid, or its modified form or derivative, or a pharmaceutically acceptable salt thereof according to [3B] above, wherein the inflammatory bowel disease is ulcerative colitis.

[5B] A D-amino acid, or its modified form or derivative, or a pharmaceutically acceptable salt thereof according to any one of [1A] to [4A] above, wherein the hepatitis is cholangitis.

[6B] A D-amino acid, or its modified form or derivative, or a pharmaceutically acceptable salt thereof according to [5B] above, wherein the cholangitis is primary sclerosing cholangitis (PSC).

[1C] A method for treatment and/or prevention of enteritis and/or hepatitis in a patient, wherein the method includes administration of an effective dose of a D-amino acid, or its modified form or derivative, or a pharmaceutically acceptable salt thereof to the patient, and the method inhibits development of symptoms or alleviates symptoms of enteritis and/or hepatitis in the patient.

[2C] The method according to [1C] above, wherein the D-amino acid is one or more selected from the group consisting of D-Ala, D-Pro, D-Gln, D-Val, D-Leu, D-Phe, D-Lys, D-Trp,

D-Glu, D-Ser and D-Asp.

[3C] The method according to [1C] or [2C] above, wherein the enteritis is inflammatory bowel disease.

[4C] The method according to [3C] above, wherein the inflammatory bowel disease is ulcerative colitis.

[5C] The method according to any one of [1C] to [4C] above, wherein the symptoms of enteritis are diarrhea, bloody stool and/or body weight loss.

[6C] A method according to any one of [1C] to [5C] above, wherein the hepatitis is cholangitis.

[7C] The method according to [6C] above, wherein the cholangitis is primary sclerosing cholangitis (PSC).

The use of a D-amino acid, or its modified form or derivative, or a pharmaceutically acceptable salt thereof for production of a drug for treatment and/or prevention of enteritis and/or hepatitis.

[2D] The use according to [1D] above, wherein the D-amino acid is one or more selected from the group consisting of D-Ala, D-Pro, D-Gln, D-Val, D-Leu, D-Phe, D-Lys, D-Trp, D-Glu, D-Ser and D-Asp.

[3D] The use according to [1D] or [2D] above, wherein the enteritis is inflammatory bowel disease.

[4D] The use according to [3D] above, wherein the inflammatory bowel disease is ulcerative colitis.

[5D] The use according to any one of [1D] to [4D] above, wherein the hepatitis is cholangitis.

[6D] The use according to [6D] above, wherein the cholangitis is primary sclerosing cholangitis (PSC).

[1E] A drug for treatment and/or prevention of hepatitis or a food composition for inhibition of hepatitis, which comprises a D-amino acid, or its modified form or derivative, or a pharmaceutically acceptable salt thereof, as an active ingredient.

[2E] A drug or a food composition for inhibition of hepatitis according to [1E] above, wherein the D-amino acid is one or more selected from the group consisting of D-Ala, D-Pro, D-Gln, D-Val, D-Leu, D-Phe, D-Lys, D-Trp, D-Glu, D-Ser and D-Asp.

[3E] A drug or a food composition for inhibition of hepatitis according to [1E] or [2E] above, wherein the hepatitis is cholangitis.

[4E] A drug or a food composition for inhibition of hepatitis according to [3E] above, wherein the cholangitis is primary sclerosing cholangitis (PSC).

[1F] A method for treatment and/or prevention of hepatitis in a patient, wherein the method includes administration of an effective dose of a D-amino acid, or its modified form or derivative, or a pharmaceutically acceptable salt thereof to the patient, and the method inhibits development of symptoms or alleviates symptoms of hepatitis in the patient.

[2F] The method according to [1C] above, wherein the D-amino acid is one or more selected from the group consisting of D-Ala, D-Pro, D-Gln, D-Val, D-Leu, D-Phe, D-Lys, D-Trp, D-Glu, D-Ser and D-Asp.

[3F] The method according to [1F] or [2F] above, wherein the hepatitis is cholangitis.

[4F] The method according to [3F] above, wherein the cholangitis is primary sclerosing cholangitis (PSC).

[1G] The use of a D-amino acid, or its modified form or derivative, or a pharmaceutically acceptable salt thereof for production of a drug for treatment and/or prevention of hepatitis.

[2G] The use according to [1G] above, wherein the D-amino acid is one or more selected from the group consisting of D-Ala, D-Pro, D-Gln, D-Val, D-Leu, D-Phe, D-Lys, D-Trp, D-Glu, D-Ser and D-Asp.

[3G] The method according to [1G] or [2G] above, wherein the hepatitis is cholangitis.

[4G] The use according to [3G] above, wherein the cholangitis is primary sclerosing cholangitis (PSC).

[1H] A method for assisting diagnosis of enteritis and/or hepatitis in a patient, wherein the amount of D-amino acid in the intestinal tract is used as an index.

[2H] The method according to [1H] above, wherein the amount of D-amino acid in feces of the patient is used as the index.

[3H] The method according to [1H] or [2H] above, wherein the D-amino acid is one or more selected from the group consisting of D-Ala, D-Pro, D-Gln, D-Val, D-Leu, D-Phe, D-Lys, D-Trp, D-Glu, D-Ser and D-Asp.

[4H] The method according to any one of [1H] to [3H] above, wherein the enteritis is inflammatory bowel disease.

[5H] The method according to [4H] above, wherein the inflammatory bowel disease is ulcerative colitis.

[6H] A method according to any one of [1H] to [5H] above, wherein the hepatitis is cholangitis.

[7H] The method according to [6H] above, wherein the cholangitis is primary sclerosing cholangitis (PSC).

### Advantageous Effects of Invention

According to the invention it is possible to provide a novel drug and food for treatment and/or prevention of enteritis and/or hepatitis, and a method for treatment or prevention of enteritis and/or hepatitis. The invention can also provide a method for assisting diagnosis of enteritis and/or hepatitis in a patient.

### BRIEF DESCRIPTION OF DRAWINGS

FIG. 1 is a set of diagrams showing an example of the invention, with the enteritis-inhibiting effect of D-amino acids in DSS-induced colitis model mice. FIG. 1A: Experiment schedule; FIG. 1B: Progressive change in body weight; FIG. 1C: DAI (disease activity index) score at 7th day after start of DSS administration; FIG. 1D: Proportion of IL-17 positive cells in large intestine at 7th day after start of DSS administration.

FIG. 2 is a set of diagrams showing an example of the invention, with the enteritis-inhibiting effect of D-amino acids in immunodeficient mice (RAG2KO). FIG. 2A: Progressive change in body weights of immunodeficient mice (left) and body weights at 8th day after start of DSS administration (right); FIG. 2B: DAI score at 8th day after start of DSS administration.

FIG. 3 is a set of diagrams showing an example of the invention, with the enteritis-inhibiting effect of different D-amino acids. FIG. 3A: Experiment schedule; FIG. 3B: Body weight at 8th day after start of DSS administration; FIG. 3C: DAI score at 8th day after start of DSS administration.

FIG. 4 shows the proportion of D-amino acids and L-amino acids in feces of healthy persons and inflammatory bowel disease (IBD) patients.

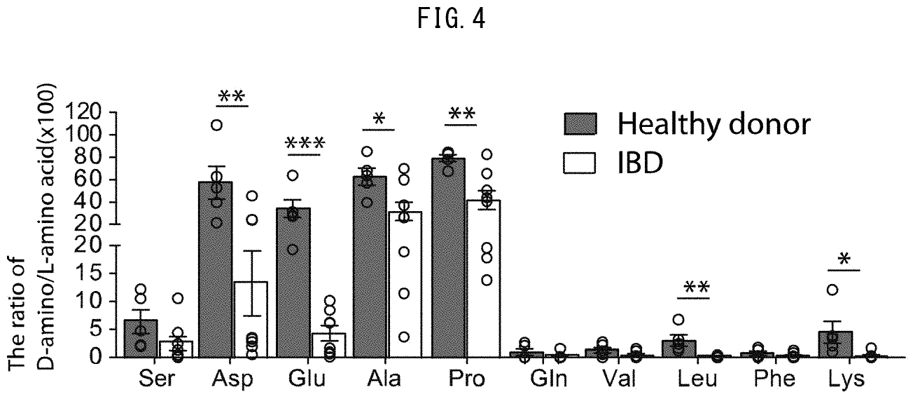

FIG. 5 is a set of diagrams showing the inflammation suppressing and preventing effect by administration of D-amino acids to liver disease model mice with concurrent ulcerative colitis.

### DESCRIPTION OF EMBODIMENTS

A preferred embodiment of the invention will now be explained in detail with reference to the accompanying drawings, with the understanding that the explanation is not necessarily limitative. The object, features, advantages and ideas inherent in the present invention will be apparent to a person skilled in the art based on the description herein, allowing a person skilled in the art to easily reproduce the invention based on the description. The following embodiments and concrete examples of the invention are preferred embodiments of the invention provided for the purpose of illustration and explanation, and are not intended to limit the invention in any way. Various modifications and embellishments such as are within the intent and scope of the invention as disclosed herein will also be apparent to a person skilled in the art based on the following description.

<D-Amino Acids>

For the purpose of the disclosure, D-amino acids are optical isomers of L-amino acids, and include D-Ala (D-alanine), D-Arg (D-arginine), D-Asn (D-asparagine), D-Asp (D-aspartic acid), D-Cys (D-cysteine), D-Gln (D-glutamine), D-Glu (D-glutamic acid), D-His (D-histidine), D-Ile (D-isoleucine), D-Leu (D-leucine), D-Lys (D-lysine), D-Met (D-methionine), D-Phe (D-phenylalanine), D-Pro (D-proline), D-Ser (D-serine), D-Thr (D-threonine), D-Trp (D-tryptophan), D-Tyr (D-tyrosine) and D-Val (D-valine), as well as their modified forms or derivatives, and their pharmaceutically or food-acceptable salts. D-Amino acid modified forms or derivatives include D-α-amino-n-butyric acid, D-homophenylalanine, D-allo-isoleucine, D-norleucine, D-norvaline and D-ornithine, for example, and derivatives also include D-phenylglycine as a derivative of glycine which does not have an optical isomer.

According to the invention, a pharmaceutically or food-acceptable salt of a D-amino acid may be in the form of any salt such as an acidic salt, basic salt or amphoteric salt, and may be any salt that is physiologically acceptable, such as a hydrochloride, sulfuric acid salt, nitric acid salt, sodium salt, potassium salt, calcium salt or ammonium salt.

According to the invention, the D-amino acid modified form or derivative is one with altered hydrophobic or electrostatic properties, or that can increase D-amino acid concentration in blood or tissue after administration. Any D-amino acid modified form or derivative may be used so long as it allows administration of the optimal amount of D-amino acid concentration in blood or tissue.

An example of a D-amino acid derivative is a compound in which the carboxy group, amino group or hydroxyl group of the D-amino acid is protected or substituted. The carboxy group may be esterified or amidated. An amino group may be amidated. Hydroxyl groups may be etherified or esterified. Examples of D-amino acid modified forms or derivatives that are D-alanine modified forms or derivatives include D-alanine methyl ester and D-alanine ethyl ester, as well as D-alanine-containing peptides such as dipeptides, tripeptides, oligopeptides or polypeptides. Examples of D-amino acid modified forms or derivatives that are D-serine modified forms or derivatives include D-serine methyl ester and D-serine ethyl ester, as well as D-serine-containing peptides such as dipeptides, tripeptides, oligopeptides or polypeptides.

When a peptide is used, it may consist of a D-amino acid alone as the active ingredient, or it may comprise other amino acids such as alanine, glycine, valine, leucine, isoleucine, threonine, cysteine, methionine, aspartic acid, glutamic acid, asparagine, glutamine, lysine, arginine, phenylalanine, tyrosine, tryptophan or histidine either in the D-form or L-form, in addition to the D-amino acid as the active ingredient.

### <Enteritis>

For the purpose of the disclosure, enteritis is a general term for diseases that present inflammation of the intestinal mucosa. The specific disease is not particularly restricted and may be infectious enteritis (for example, enteritis caused by, e.g., Staphylococcus, Vibrio parahaemolyticus, Salmonella or Vibrio cholerae), or inflammatory bowel disease including Crohn disease and ulcerative colitis.

Inflammatory bowel disease (IBD) includes Crohn disease and ulcerative colitis, and it is a pathology with repeating relapse and remission, characterized by chronic inflammation of the gastrointestinal tract with diarrhea and abdominal pain.

Ulcerative colitis (UC) is an inflammatory disease of the large intestine with sores and ulcers in the large intestinal mucosa. Its characteristic symptoms are diarrhea and abdominal pain, either with or without bloody stool. Lesions spread out in an ascending and continuous manner from the rectum, maximally reaching from the rectum throughout the colon. Ulcerative colitis is classified as follows, depending on the extent of the lesion and progression.

1) Classification based on extent of lesion: Pancolitis, left-sided colitis, proctitis.
2) Classification of disease stage: Active, remissive.
3) Classification based on severity: Mild, moderate, severe, very severe.
4) Classification based on clinical progression: Recrudescent remissive, chronic continuous, acute fulminating, initial colitis.

The UC-DAI (Disease active index), including stool frequency, bloody stool volume, mucosal membrane observation and overall evaluation by a physician, is used to determine the severity of ulcerative colitis or for monitoring improvement by drugs.

Ulcerative colitis is a designated intractable disease, and conventionally, for mild cases, symptoms (inflammation) have been controlled by administration of a 5-aminosalicylic acid formulation which improves the symptoms of inflammatory bowel disease via an active oxygen removal effect or leukotriene production inhibiting effect, or in the absence of an observable effect, administration of steroids or white blood cell component removal therapy has been attempted, with administration of biopreparations such as immunomodulators or TFα being used in cases where symptoms are still not abated. Large intestine extraction surgery has been used when no effect is observed even with these measures, and therefore the development of new treatment agents is strongly desired. The cause of ulcerative colitis is thought to be abnormal autoimmune reaction or dietary habits, but it is still not fully understood.

### <Hepatitis>

Throughout the present disclosure, hepatitis is used as a general term for diseases associated with inflammation of hepatocytes due to any cause. Different types depending on the cause include viral hepatitis, alcoholic hepatopathy, non-alcoholic steatohepatitis, drug-induced liver disorder, autoimmune hepatitis, and various types of cholangitis, with pathologies exhibiting acute or chronic symptoms.

### <Cholangitis>

For the purpose of the disclosure, cholangitis is a general term for diseases that present inflammation of the bile duct. Specific diseases include, but are not particularly limited to, acute cholangitis resulting from bacterial infection when stones are present in the common bile duct, or when incarcerated stones are present, or when there is a stricture in the bile duct; and primary sclerosing cholangitis (PSC). Primary sclerosing cholangitis (PSC), in particular, is a condition characterized by spotted inflammation, fibrosis and stenosis of the bile duct, the cause of which is still unclear. About 80% of patients suffering from primary sclerosing cholangitis (PSC) also concurrently suffer from inflammatory bowel disease, and particularly ulcerative colitis. According to one aspect, the invention can treat or prevent cholangitis such as primary sclerosing cholangitis (PSC).

<Drug for Treatment and/or Prevention of Enteritis and/or Hepatitis, and Method for its Administration>

One aspect of the invention is a drug (or pharmaceutical composition) for treatment and/or prevention of enteritis and/or hepatitis, which comprises a D-amino acid, or its modified form or derivative, or a pharmaceutically acceptable salt thereof. The drug according to one aspect of the invention may also contain a pharmacologically acceptable carrier, diluent or excipient in addition to the D-amino acid, or its modified form or derivative, or a pharmaceutically acceptable salt thereof. The drug according of the invention may further contain an anti-enteritis agent or anti-hepatitis agent in addition to the D-amino acid, or its modified form or derivative, or a pharmaceutically acceptable salt thereof. The drug of the invention may be formulated in a dosage form selected to be suitable for the route of administration. The dosage form may be designed as a tablet, capsule, liquid drug, powdered drug, granules or a chewable agent for use in oral administration, or as an injection, powdered drug or infusion preparation for parenteral administration. These formulations may also include various types of adjuvants such as carriers or other auxiliary agents that are used in drugs, including stabilizers, antiseptic agents, soothing agents, flavorings, taste correctives, aromatics, emulsifiers, fillers and pH adjustors, in ranges that do not interfere with the effect of the composition of the invention. In order to improve or maintain the effect of the drug, the optical purity of the D-amino acid is preferably 50% or greater and more preferably 90% or greater.

One aspect of the invention is a method for administering a drug for treatment and/or prevention of enteritis and/or hepatitis, which comprises a D-amino acid, or its modified form or derivative, or a pharmaceutically acceptable salt thereof. Treatment of enteritis and/or hepatitis includes its remission (i.e. a state where symptoms have subsided). Enteritis symptoms include diarrhea, bloody stool and/or body weight decrease, while continued hepatitis may progress to hepatic fibrosis, hepatic cirrhosis or hepatocellular carcinoma.

The drug may be provided in a form suited for local administration (skin, inhalation, enema, eye drop, ear drop, nasal or intravaginal), enteral administration or parenteral administration (intravenous, transarterial, transdermal or intramuscular injection), with enteral administration being preferred. Enteral administration includes oral administration, tube administration and enema administration. Tube administration includes administration through a nasal stomach tube or gastric fistula, or duodenal fistula. Enema administration includes administration using a suppository or enema. In any case there is no particular restriction on the dosage form of the drug, which may be liquid or solid, and produced by common technical knowledge to those skilled in the art. The specific method of administration is also not particularly restricted, and it may be suitable administration according to common technical knowledge to those skilled in the art.

The administered individual is not particularly restricted but is preferably a human or a vertebrate other than a human, more preferably a mammal such as a human or mouse, and even more preferably a human.

The dose of the D-amino acid as a drug or the amount of consumption of the D-amino acid as a food composition may be set to any amount from the viewpoint of exhibiting the effect of treating or preventing enteritis and/or hepatitis, but it is preferably adjusted as appropriate for the concentration desired in the intestinal tract and/or liver, in light of the following reports in regard to D-serine.

D-Serine has been reported to produce nephrotoxicity with intraperitoneal administration to rats (NPL (1): Alexander W. K., Am J Physiol Renal Physiol 2007, 293, F382-F390; NPL (2): M. Maekawa et al., Chem. Res. Toxicol. 2005, 18, 1678-1682; NPL (3): M. Orozco-Ibarra et al., Toxicology 229 (2007) 123-135; NPL (4): R. E. Williams et al., Toxicology 207 (2005) 179-190; NPL (5): R. E. Williams, E. A. Lock, Toxicology 201 (2004) 231-238). In these publications it is disclosed that administration in doses exceeding 250 mg/kg, such as 800 mg/kg (NPL (2)), 400 mg/kg (NPL (3)) or 250 mg/kg (NPLs (4) and (5)) causes nephrotoxicity due to oxidative stimulation or DAO action. Specifically, it is thought that D-serine is metabolized by the action of D-amino acid oxidase (DAO) in the proximal tubules, and that the active oxygen species generated by this reaction causes cell damage and leads to necrosis.

In the research in rats in NPL1, the renal effects of administering D-serine at 0.25, 0.76, 2.54 and 7.6 mmol/kg by intraperitoneal administration are examined. Since the molecular weight of D-serine is 105, these doses correspond to 26 mg/kg, 80 mg/kg, 267 mg/kg and 800 mg/kg, respectively. When D-serine was intraperitoneally administered at such concentrations, there was no effect on amino acid excretion and no toxicity was exhibited after 2 hours with 26 mg/kg or 80 mg/kg, but with 267 mg/kg and 800 mg/kg the amino acid excretion significantly increased and toxicity of D-serine on the kidneys was exhibited. When amino acid excretion after 4 hours was examined, no toxicity was exhibited with 26 mg/kg but very weak toxicity was exhibited with 80 mg/kg. Therefore, the non-toxic level (NOAEL) in rats may be determined to be 25, 30, 40 or 50 mg/kg. Thus, setting the dose of D-serine to the NOAEL, and also setting a safety factor of 10 in consideration of the difference in species, 2.5 to 5.0 mg/kg/day may be set as the upper limit for dosage. A different safety factor may also be set if desired. The upper limit for the dosage is preferably 5.0 mg/kg/day, more preferably 4.0 mg/kg/day, even more preferably 3.0 mg/kg/day and most preferably 2.5 mg/kg.

In the Examples described below, mice were freely provided with drinking water containing 1% D-serine until the blood D-serine concentrations reached 100 nmol/mL, since at this concentration D-serine was able to exhibit a therapeutic effect on enteritis and hepatitis. While it is necessary to take species differences into consideration, but without being limited to theory, the effect of the invention can be exhibited as a therapeutic effect on enteritis and hepatitis with a D-serine blood concentration of 1 nmol/mL to 1 μmol/mL. The blood concentration of administered D-serine is preferably 5 nmol/mL or greater, more preferably 10 nmol/mL or greater and even more preferably 50 nmol/mL or greater. The D-serine blood concentration is also preferably 1 μmol/mL or lower, more preferably 0.5 μmol/mL or lower and even more preferably 0.1 μmol/mL or lower.

<Food for Inhibition of Enteritis and/or Hepatitis>

One aspect of the invention is a food for inhibition of enteritis and/or hepatitis, which comprises a D-amino acid, or its modified form or derivative, or a food-acceptable salt thereof, or a food containing the same as an additive. A food for inhibition of enteritis and/or hepatitis is a food indicated for ingestion by a patient suffering from enteritis and/or hepatitis or a patient with risk of suffering from enteritis and/or hepatitis. The form of the food is not particularly restricted, and it may be added to a routine meal or may be a supplement or supplement food. For the purpose of the present disclosure, foods also include beverages. Such foods may also be provided as functional foods or health foods. A D-amino acid, or its modified form or derivative, or a food-acceptable salt thereof, or a food material comprising the same, may be incorporated into any food product. In order to improve or maintain the effect of the food product, the optical purity of the D-amino acid is preferably 50% or greater and more preferably 90% or greater.

Symptoms of enteritis and/or hepatitis can be improved by ingesting the food for inhibition of enteritis and/or hepatitis. When used as an intestinal regulator, for example, enteritis and/or hepatitis can also be prevented.

<Method for Assisting Diagnosis of Enteritis and/or Hepatitis>

One aspect of the invention provides a method for assisting diagnosis of enteritis and/or hepatitis in a patient, wherein the amount of D-amino acid in the intestinal tract is used as an index. As a result of avid research, the present inventors have found that the amount of D-amino acid varies in the feces of healthy persons and enteritis patients, and particularly inflammatory bowel disease (IBD) patients. It was then found that by measuring the amounts of D-amino acids in the intestinal tracts of patients, it is possible to judge the pathological condition of patient enteritis, such as inflammatory bowel disease and especially ulcerative colitis, for use in diagnosis. The pathological condition of enteritis can be assessed by measuring the amount of D-amino acids in enteral contents such as feces. This index is not limited to assessing the pathological condition, and may also be used for diagnosis of disease or prognosis, or assessment of the effects of treatment or administered drugs. Numerical values calibrated with L-amino acids may also be used in this case. The D-amino acids to be used for assessment may be one or more D-amino acids selected from the group consisting of D-Ala, D-Pro, D-Gln, D-Val, D-Leu, D-Phe, D-Lys, D-Trp, D-Glu, D-Ser and D-Asp, and they may also be used in combination. The amounts of L-amino acids with respect to selected D-amino acids may also be measured and assessment made using the calibrated values, or assessment may be made by the ratio of D-amino acids to L-amino acids. The invention may also assess the pathological condition of hepatitis such as cholangitis, and especially primary sclerosing cholangitis (PSC), that is concurrent with enteritis such as inflammatory bowel disease, and especially ulcerative colitis, for use in diagnosis.

A patient whose condition of enteritis and/or hepatitis has been assessed by the method of the invention can be treated by administration or ingestion of a drug or a food composition for treatment and/or prevention of enteritis and/or hepatitis as already described for another aspect.

In addition, a patient whose condition of enteritis and/or hepatitis has been assessed by the method of the invention may be treated by a publicly known treatment method used for treatment of enteritis, such as administration of an aminosalicylic acid salt formulation (such as mesalazine (5-ASA)), an anti-TNF-α antibody formulation (such as infliximab), an anti-IL-12/23p40 antibody formulation (such as ustekinumab), an anti-α4β7 integrin antibody formulation (such as vedolizumab), a JAK inhibitor (such as tofacitinib citrate), a steroid, an immunomodulator (such as a thioprine formulation: azathioprine), an immunosuppressant drug (such as a calcineurin inhibitor: cyclosporin), a glycyrrhizin formulation, a synthetic disaccharide, an antibacterial agent, an antihypertensive drug, a diuretic drug, an albumin formulation, a carnitine formulation, an opioid κ-receptor agonist, carnitine, acetylcysteine, a resin, a dyslipidemia therapeutic agent (such as a statin, small intestine cholesterol transporter inhibitor or fibrate), a thiazoline derivative, an oil-based contrast agent, a gallbladder drug (such as ursodeoxycholic acid), a protease inhibitor, antithrombin, an alcohol dependency therapeutic agent (such as cyanamide), an HA vaccine, an anti-HB human immunoglobulin, an HB vaccine or an anti-hepatitis virus drug (such as an interferon formulation, an RNA polymerase inhibitor or a protease inhibitor), nutrition therapy (such as ingestion of an elemental nutritional supplement, a digestive nutritional supplement or semi-digestive nutritional supplement), blood cell component removal therapy, or surgical operation.

The present invention will now be explained in greater detail through Examples, with the understanding that the Examples are in no way limitative on the invention.

### EXAMPLES

### Example 1: Enteritis-Inhibiting Effect of D-Amino Acids in DSS-Induced Colitis Model

### 1. Method

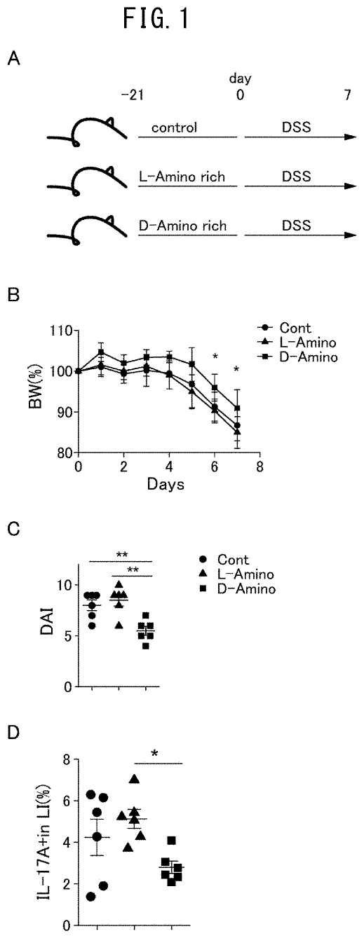

Mice (B6CL57) given free access for 3 weeks to drinking water containing 5 different D-amino acids (1% D-serine, 0.75% D-glutamic acid, 1% D-asparagine, 1% D-tryptophan and 1% D-alanine) (indicated as D-Amino in the diagram); or drinking water containing 5 different L-amino acids (1% L-serine, 0.75% L-glutamic acid, 1% L-asparagine, 1% L-tryptophan and 1% L-alanine) (indicated as L-Amino in the diagram), and mice given free access for 3 weeks to normal drinking water (control), were chemically induced to have colitis by free access to drinking a 2% DSS (dextran sulfate sodium) solution for 7 days thereafter, and the severity of enteritis (body weight and pathology score (DAI score)) was measured (FIGS. 1B and C).

The DAI scores were assigned for the following: diarrhea symptom (normal: 0, soft feces: 1, mild diarrhea: 2, moderate diarrhea: 3, severe diarrhea: 4); bloody stool symptom (normal: 0, mild bloody stool: 1, moderate bloody stool: 2, severe bloody stool: 3, severe bloody stool with anal bleeding: 4); and body weight reduction (None: 0, 1% to 5%: 1, 5% to 10%: 2, 10% to 20%: 3, ÷20%: 4), and the total values were calculated (minimum 0 points, maximum 12 points), for each mouse.

The proportion of Th17 cells, which are IL-17-positive T cells, were measured as inflammatory cells in the large intestine (FIG. 1D).

### 2. Results

In the mouse group given drinking water containing D-amino acids, body weight decrease was significantly inhibited after the 6th day, as shown in FIG. 1B, and the DAI scores were significantly lower on the 7th day, as shown in FIG. 1C, compared to the mouse group given drinking water containing L-amino acids and the mouse group given normal drinking water.

Also, as shown in FIG. 1D, the proportion of IL-17-positive Th17 cells in the large intestine on the 7th day were significantly lowered in the mouse group given drinking water containing D-amino acids compared to the mouse group given drinking water containing L-amino acids. Th17 cells which produce IL-17 in the intestinal tract are a cell population that increases with inflammatory bowel disease, and they are inflammatory cells. Th17 cells therefore increase in intestinal tracts with inflammation, and Th17 cells decrease in intestinal tracts with inhibited inflammation. Inflammation caused by ingestion of DSS was thus inhibited in the mouse group given drinking water containing D-amino acids.

Aggravation of enteritis was thus significantly inhibited in the mouse group given drinking water containing D-amino acids.

### Example 2: Enteritis-Inhibiting Effect of D-Amino Acids in Immunodeficient Mice

### 1. Method

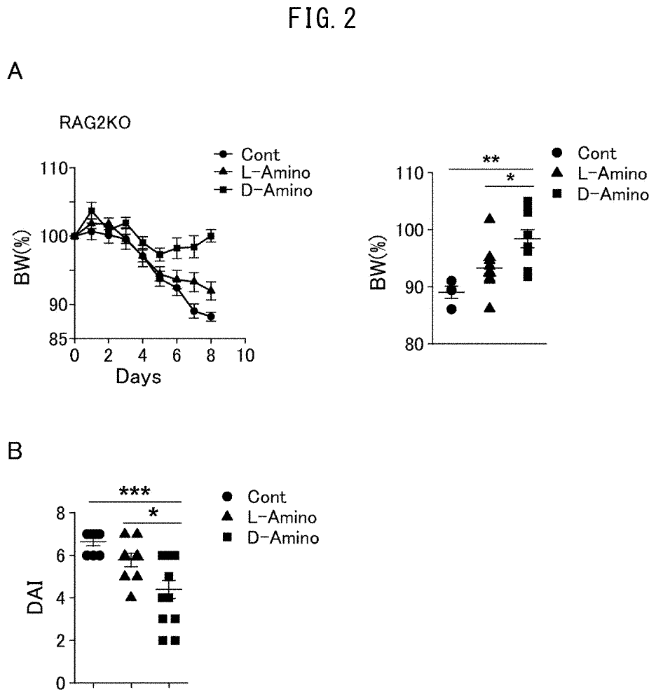

Immunodeficient mice (RAG2-deficient mice: RAG2KO) given free access for 3 weeks to drinking water containing 5 different D-amino acids (1% D-serine, 0.75% D-glutamic acid, 1% D-asparagine, 1% D-tryptophan and 1% D-alanine) or 5 different L-amino acids (1% L-serine, 0.75% L-glutamic acid, 1% L-asparagine, 1% L-tryptophan and 1% L-alanine), and immunodeficient mice given free access for 3 weeks to normal drinking water, were chemically induced to have colitis by free access to drinking a 2% DSS solution for 7 days, and the severity of enteritis (body weight and pathology score (DAI score)) was measured (FIGS. 2A and B).

### 2. Results

For the RAG2KO mice as well, the results obtained were similar to Example 1, in that the mouse group given drinking water containing D-amino acids had significantly inhibited aggravation of enteritis compared to the mouse group given drinking water containing L-amino acids and the mouse group given normal drinking water. This indicates that D-amino acids have an enteritis-inhibiting effect even in mice without acquired immune function.

### Example 3: Enteritis-Inhibiting Effect of Single Species D-Amino Acids in DSS-Induced Colitis Model Mice

### 1. Method

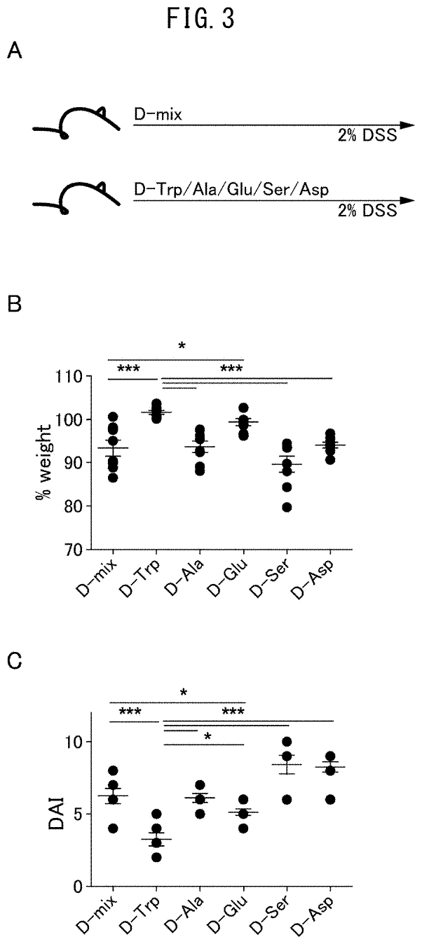

Mice (B6CL57) given free access for 3 weeks to drinking water containing 5 different D-amino acids (1% D-serine, 0.75% D-glutamic acid, 1% D-asparagine, 1% D-tryptophan and 1% D-alanine) or drinking water containing one D-amino acid alone (1% D-serine, 0.75% D-glutamic acid, 1% D-asparagine, 1% D-tryptophan or 1% alanine), were chemically induced to have colitis by free access to drinking a 2% DSS solution for 7 days, and the severity of enteritis (body weight and pathology score (DAI score)) was measured (FIG. 3A).

### 2. Results

For both the body weight reduction suppressing effects and DAI scores, the drinking water containing the D-amino acids D-Ser, D-Ala and D-Asp had enteritis-inhibiting effects equivalent to drinking water containing all five D-amino acids, while the drinking waters containing the D-amino acids D-Trp and D-Glu significantly inhibited enteritis more than the drinking water containing all five D-amino acids (FIGS. 3B and C).

### Example 4: Pathology Index Based on D-Amino Acids in Feces of Inflammatory Bowel Disease (IBD) Patients

Feces harvested from healthy persons and inflammatory bowel disease patients were homogenized with MilliQ water, methanol was added and the mixture was centrifuged to modify the extracted amino acids with NBD-F, and the data from separation and quantification by liquid chromatography were provided for analysis. The D-amino acids in the feces of the IBD patients (calibrated using L-amino acids) were characteristically lower than in healthy persons, indicating that D-amino acid levels in feces are useful for assisting in ascertaining pathology, setting drug dose and assessing therapeutic or drug effect.

### Example 5: Inflammation-Inhibiting Effect in DDC-Induced Liver Disease Model

### 1. Method

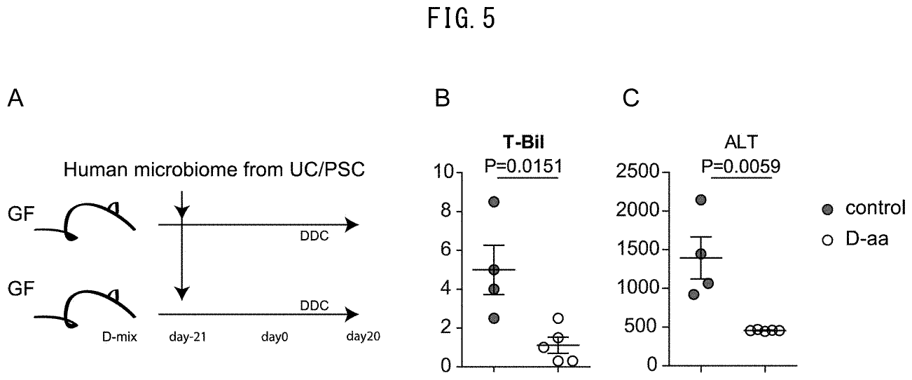

Using model mice obtained by transplanting feces from patients with ulcerative colitis (UC) and primary sclerosing cholangitis (PSC) into aseptic mice (GF) given free access to drinking water containing 5 different D-amino acids (1% D-serine, 0.75% D-glutamic acid, 1% D-asparagine, 1% D-tryptophan and 1% D-alanine) (indicated as D-mix in the diagram), and induced to have cholangitis using 3,5-diethoxycarbonyl-1,4-dihydrocollidine (DDC), and in the same model mice given free access to normal drinking water (FIG. 5A), the total bilirubin (T-Bill) and aminotransferase (ALT) were measured as blood liver disease markers.

### 2. Results

In the mouse group given drinking water containing D-amino acids, the liver disease markers were significantly reduced and hepatitis was inhibited and prevented, compared to the mouse group given normal drinking water (FIGS. 5B and C).

### INDUSTRIAL APPLICABILITY

According to the invention it is possible to provide a novel drug and food for treatment and/or prevention of enteritis and/or hepatitis, and a method for treatment or prevention of enteritis and/or hepatitis.

1. A drug for treatment and/or prevention of enteritis and/or hepatitis,
which comprises a D-amino acid, or its modified form or derivative, or a pharmaceutically acceptable salt thereof, as an active ingredient.

2. A drug according to claim 1, wherein the D-amino acid is one or more selected from the group consisting of D-Ala, D-Pro, D-Gln, D-Val, D-Leu, D-Phe, D-Lys, D-Trp, D-Glu, D-Ser and D-Asp.

3. A drug according to claim 1 or 2, wherein the enteritis is inflammatory bowel disease.

4. A drug according to claim 3, wherein the inflammatory bowel disease is ulcerative colitis.

5. A drug according to any one of claims 1 to 4, wherein the hepatitis is cholangitis.

6. A drug according to claim 5, wherein the cholangitis is primary sclerosing cholangitis (PSC).

7. The use of a D-amino acid, or its modified form or derivative, or a pharmaceutically acceptable salt thereof for production of a drug for treatment and/or prevention of enteritis and/or hepatitis.

8. The use according to claim 7, wherein the D-amino acid is one or more selected from the group consisting of D-Ala, D-Pro, D-Gln, D-Val, D-Leu, D-Phe, D-Lys, D-Trp, D-Glu, D-Ser and D-Asp.

9. The use according to claim 7 or 8, wherein the enteritis is inflammatory bowel disease.

10. The use according to claim 9, wherein the inflammatory bowel disease is ulcerative colitis.

11. The use according to any one of claims 7 to 10, wherein the hepatitis is cholangitis.

12. The use according to claim 11, wherein the cholangitis is primary sclerosing cholangitis (PSC).

13. A food composition for inhibition of enteritis and/or hepatitis, which comprises a D-amino acid, or its modified form or derivative, or a food-acceptable salt thereof, as an active ingredient.

14. A food composition according to claim 13, wherein the D-amino acid is one or more selected from the group consisting of D-Ala, D-Pro, D-Gln, D-Val, D-Leu, D-Phe, D-Lys, D-Trp, D-Glu, D-Ser and D-Asp.

15. A food composition according to claim 13 or 14, wherein the enteritis is inflammatory bowel disease.

16. A food composition according to claim 15, wherein the inflammatory bowel disease is ulcerative colitis.

17. A food composition according to any one of claims 13 to 16, wherein the hepatitis is cholangitis.

18. A food composition according to claim 17, wherein the cholangitis is primary sclerosing cholangitis (PSC).

19. A food comprising a food composition according to any one of claims 13 to 18.

20. A method for assisting diagnosis of enteritis and/or hepatitis in a patient, wherein the amount of D-amino acid in the intestinal tract is used as an index.

21. The method according to claim 20, wherein the amount of D-amino acid in feces of the patient is used as the index.

22. The method according to claim 20 or 21, wherein the D-amino acid is one or more selected from the group consisting of D-Ala, D-Pro, D-Gln, D-Val, D-Leu, D-Phe, D-Lys, D-Trp, D-Glu, D-Ser and D-Asp.

23. The method according to any one of claims 20 to 22, wherein the enteritis is inflammatory bowel disease.

24. The method according to claim 23, wherein the inflammatory bowel disease is ulcerative colitis.

25. The method according to any one of claims 20 to 24, wherein the hepatitis is cholangitis.

26. The method according to claim 25, wherein the cholangitis is primary sclerosing cholangitis (PSC).
